(19) United States
(12) Patent Application Publication
(10) Pub. No.: US 2026/0208152 A1
(43) Pub. Date: Jul. 23, 2026

(54) MOLECULAR SIEVE WITH POINT-SIEVING STRUCTURE, ITS PREPARATION METHOD, AND APPLICATION IN ADSORPTIVE SEPARATION OF PROPYLENE AND PROPANE THEREOF

(71) Applicant: ZHEJIANG UNIVERSITY, HANGZHOU, Zhejiang Province (CN)

(72) Inventors: HUABIN XING, HANGZHOU, ZHEJIANG PROVINCE (CN); JIYU CUI, HANGZHOU, ZHEJIANG PROVINCE (CN); LIFENG YANG, HANGZHOU, ZHEJIANG PROVINCE (CN); XILI CUI, HANGZHOU, ZHEJIANG PROVINCE (CN)

(21) Appl. No.: 19/141,838

(22) PCT Filed: Dec. 9, 2024
(86) PCT No.: PCT/CN2024/137778

§ 371 Date: Jun. 20, 2025

(30) Foreign Application Priority Data
Dec. 12, 2023 (CN) 202311699773.9

(51) Int. Cl. B01J 20/22; B01J 20/34; C07C 7/13; C07C 9/08; C07C 11/06

(52) U.S. Cl. CPC B01J 20/223; B01J 20/3425; B01J 20/3458; B01J 20/3483; B01J 20/3491; C07C 7/13; C07C 9/08; C07C 11/06; B01J 2220/56; B01J 2220/60

(57) ABSTRACT

The present invention discloses a molecular sieve with a point-sieving structure, its preparation method, and its application in the adsorptive separation of propylene and propane. The molecular sieve is formed through coordination bonds among 1,2-ethanedisulfonate anions, 4,4′-dipyridyl sulfide, and Cu²⁺, with the chemical formula [MSL₂]_{∞}, where M represents Cu²⁺, S denotes the 1,2-ethanedisulfonate anion, L denotes 4,4′-dipyridyl sulfide, and ∞ indicates that the molecular sieve is spatially extended from multiple MSL₂ structural units. The molecular sieve features localized point-like pore constrictions. Preparation Method: A Cu²⁺ source, 1,2-ethanedisulfonate salt, and 4,4′-dipyridyl sulfide are mixed and reacted in a reaction solvent to obtain the molecular sieve. The present invention exhibits outstanding advantages, including excellent material stability, high adsorption selectivity, large adsorption capacity, rapid adsorption kinetics, and ease of regeneration, demonstrating significant industrial application.

### TECHNICAL FIELD

The present invention relates to the technical field of molecular sieves and chemical separation technologies, and in particular to a molecular sieve with a point-sieving structure, its preparation method, and its application in separating propylene and propane.

### BACKGROUND TECHNOLOGY

Propylene, as a cornerstone of the petrochemical industry, is one of the most produced chemicals globally. Its downstream products, including polypropylene, propylene oxide, and acrylonitrile, are widely used in plastics, pharmaceuticals, textiles, coatings, and other sectors of the national economy. Currently, industrial production of propylene primarily involves separation from light hydrocarbon mixtures derived from naphtha cracking. A key technical challenge in separating these hydrocarbons lies in the close molecular sizes and similar physical properties of ethylene/ethane and propylene/propane. However, propylene and propane exhibit highly similar structural properties, differing only slightly in unsaturation, which makes their separation both energy-intensive and technically demanding. Existing separation technologies for propylene/propane include cryogenic distillation, solvent absorption, membrane separation, and adsorptive separation. Among these, cryogenic distillation is the most mature and widely used industrially. However, due to the close boiling points and low relative volatility of propylene and propane, separation requires high pressure (~22 bar), extremely low temperatures (~160° C.), and a high reflux ratio, typically necessitating over 150 distillation trays. This has the disadvantages of high energy consumption and significant equipment investment. Solvent absorption methods suffer from issues such as large organic solvent pollution and low selectivity. Membrane separation is limited by the low selectivity of existing membranes, complex fabrication processes, and high costs, restricting its industrial application.

Adsorptive separation technology, as an energy-efficient and environmentally friendly approach, is suitable for separating structurally similar substances, such as the separation and purification of low-carbon hydrocarbon gases. However, existing adsorbent materials—including zeolites molecular sieves, activated carbon, polymers, and metal-organic frameworks (MOFs)—often struggle to precisely distinguish the subtle differences between propylene and propane molecules. Challenges such as low separation selectivity, limited adsorption capacity, poor stability, and slow adsorption kinetics hinder the feasibility of propylene/propane adsorptive separation. For instance, while 4A molecular sieve can achieve molecular sieving of propylene and propane, there is a problem that the propylene diffusion coefficient is too slow at ambient temperature (10⁻¹¹ cm²/s). Therefore, propylene and propane can only be separated by enhancing propylene diffusion at a high-temperature operation (~150° C.) (Separation Science and Technology, 2010, 45:1252-1259). MOFs, as emerging porous adsorbents, offer advantages such as large specific surface area, high pore volume, easy chemical modification of pores, and precise adjustment of pore size. However, the separation performance of the π-complexed framework materials, which are currently widely studied, Fe-MOF-74 (Science, 2012, 335 (6076): 1606-1610), has a high density of unsaturated metal sites, which can generate electrostatic interactions with the double bond π electrons of propylene, but due to its large pore size (~11 Å), its selectivity for propylene/propane mixed gas is low. By utilizing the molecular size difference between propylene (~4.0 Å) and propane (~4.3 Å) designing propylene propane molecular sieve materials with suitable pore sizes can significantly improve the separation selectivity of propylene/propane. For example, KAUST-7 (Science, 2016, 353:137-140) and Y-abtc (Advanced Materials, 2018, 30:1805088) have suitable pore sizes, which can adsorb smaller propylene molecules while excluding larger propane molecules. Nevertheless, current propylene/propane sieving materials still face challenges such as low working capacity, slow adsorption rates, and regeneration difficulties. Thus, developing novel porous materials capable of molecular sieving for propylene/propane separation—with high adsorption capacity, rapid kinetics, and facile regeneration—is critical for advancing adsorption-based separation technologies.

### SUMMARY OF INVENTION

To address the aforementioned technical challenges and existing deficiencies in the field, the present invention provides a molecular sieve with a point-sieving structure (denoted as ZU-609), constructed from rigid linear 1,2-ethanedisulfonate anions, flexible and bendable 4,4′-dipyridyl sulfide (CAS No. 37968-97-1), and Cu²⁺ metal cations via coordination bonds. When a propylene/propane mixture contacts this molecular sieve, propylene is preferentially adsorbed, enabling high-purity propylene production. By precisely tuning the pore size and channel geometry, the molecular sieve ZU-609 achieves high selectivity and capacity for propylene adsorption while excluding propane from entering the pores. The present invention exhibits outstanding advantages, including material stability, high adsorption selectivity, large adsorption capacity, rapid adsorption kinetics, and easy regeneration, demonstrating strong industrial applicability.

A molecular sieve with a point-sieving structure is formed via coordination bonds among 1,2-ethanedisulfonate anions (organic anions), 4,4′-dipyridyl sulfide (organic ligand), and Cu²⁺ (metal cation), with the chemical formula [MSL₂]_{∞}, where M represents Cu²⁺, S denotes the 1,2-ethanedisulfonate anion, L denotes 4,4′-dipyridyl sulfide, and ∞ indicates that the molecular sieve is spatially extended from multiple MSL₂ structural units.

The molecular sieve features localized point-like pore constrictions (see FIG. 1).

In the molecular sieve structure with a point-sieving structure, organic ligands coordinate with metal cations via nitrogen atoms, with all organic ligands being bidentate. Each 1,2-ethanedisulfonate anion connects two metal cations through oxygen atoms, and each 1,2-ethanedisulfonate anion is connected to two metal cations; each metal cation is connected to four distinct organic ligands and coordinated with two oxygen atoms synergistically.

The present invention prepares a new type of molecular sieve material with a point-sieving structure by combining metal cations, organic ligands, and organic anions, and the present invention achieves precise control over pore size in the molecular sieve porous material with a point-sieving structure. When propylene and propane molecules come into contact with the molecular sieve porous material ZU-609 with a point-sieving structure, since propylene has a smaller molecular size than propane, the precisely adjusted material pore size enables propylene to enter the molecular sieve porous material with a point-sieving structure while excluding propane from entering the pores, thereby showing good size sieving characteristics. At the same time, due to the structural characteristics of the molecular sieve of the present invention, the high-diffusion-energy-barrier paths formed by the diffusion of for propylene molecules within the pores is shorter (see FIG. 3).

The 1,2-ethanediyl disulfonate anion as described in the present invention can be expressed as follows:

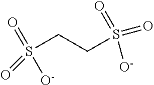

The molecular sieve with a point-sieving structure of the present invention has a sieving hole has a cross-sectional dimension of 4.2×5.1 Å², effectively excluding propane.

The flexible and bendable 4,4′-dipyridyl sulfide ligand and the rigid linear 1,2-ethanedisulfonate anion synergistically contribute to constructing the large pore cavity of the molecular sieve (see FIG. 2). The rigid pyridine rings on the ligand molecules provide structural prerequisites for forming the sieving windows.

The molecular sieve of the present invention further features a large pore cavity (7.5×8.1×11.1 Å³), which offers ample space to achieve an exceptionally high diffusion coefficient and adsorption capacity for propylene.

Due to its smaller kinetic diameter, propylene can enter the pore channels of the molecular sieve, whereas propane, with a larger kinetic diameter, is excluded. This enables efficient adsorptive separation of propylene/propane mixtures.

The present invention also provides a method for preparing the molecular sieve with a point-sieving structure. A Cu²⁺ source, 1,2-ethanedisulfonate salt, and 4,4′-dipyridyl sulfide are mixed and reacted in a reaction solvent to yield the molecular sieve with a point-sieving structure.

The reaction solvent may be water and/or an organic solvent. The organic solvent may include methanol, among others. When the reaction solvent comprises both water and an organic solvent, the volume ratio of water to organic solvent may range from 1:1 to 1:5.

The Cu²⁺ source may include at least one of chlorides, nitrate compounds, or similar substances.

The 1,2-ethanedisulfonate salt may include sodium 1,2-ethanedisulfonate, among others.

In the preparation method, the molar ratio of Cu²⁺ source, 1,2-ethanedisulfonate salt, and 4,4′-dipyridyl sulfide is Cu²⁺:1,2-ethanedisulfonate anion: 4,4′-dipyridyl sulfide=1:0.5-3:1-5.

The ratio of Cu²⁺ source to reaction solvent is 1 mol: 35-45 L.

In one embodiment, after the mixed reaction is completed, solid-liquid separation is performed. The collected solid is treated to remove solvent molecules from the pores, yielding the molecular sieve with a point-sieving structure. Solvent removal may be achieved via vacuum desorption and/or drying by purging with flowing gas (e.g., N₂ or inert gases such as He or Ar).

The synthesis of the molecular sieve with a point-sieving structure may involve solvothermal reactions, interfacial diffusion reactions via dropwise addition, or direct mixing at room temperature.

The present invention further provides the use of the molecular sieve with a point-sieving structure for propylene adsorption.

ZU-609 exhibits the following adsorption properties:
Linear adsorption isotherms, suitable for pressure swing adsorption (PSA);
Propylene diffusion coefficient at ambient temperature: 10.02×10⁻¹⁰ cm²/s;
Adsorption capacity difference between 0.1 bar and 1 bar: 2.0 mmol g⁻¹;
Propylene/propane adsorption ratio at 1 bar: 22.

Propylene adsorbs rapidly within the pore channels of the molecular sieve, enabling operation under high gas flow rates in dynamic separation experiments with propylene/propane mixtures.

As a general inventive concept, the present invention further provides a method for adsorptive separation of propylene and propane. The method employs the molecular sieve with a point-sieving structure as an adsorbent, which selectively adsorbs propylene while excluding propane from a propylene/propane mixture.

This enables efficient separation under mild operating conditions, yielding propylene gas with a purity of 97.0%-99.99%.

The contact method between the adsorbent and the propylene/propane mixture may involve fixed-bed adsorption, moving-bed adsorption, multi-tower pressure swing adsorption, or a combination thereof.

### Example of Multi-Tower Pressure Swing Adsorption (PSA):

1. A mixed gas containing propylene and propane is compressed by a compressor and fed into the first adsorption tower under constant pressure. The adsorption pressure in Tower 1 is controlled at 1 bar-10 bar, and the temperature is maintained at 25° C.-40° C. Propane-rich gas (>99.9% purity) exits directly from the outlet of Tower 1.
2. Tower 2 undergoes purging and vacuum desorption, while Towers 3 to n (n≥3) perform pressure equalization. When adsorption in Tower 1 is complete, vacuum desorption in Tower 2 is halted, and Tower 1 initiates pressure equalization with Tower 2.
3. A portion of high-purity propylene collected from the previous cycle is used for reflux purging in Tower 1, while Tower n is rapidly pressurized with product gas and enters the adsorption phase.
4. Vacuum desorption of Tower 1 is performed using a vacuum pump, with the desorption pressure controlled at 0 bar-0.1 bar. High-purity propylene (>99.5%) is recovered from the outlet, with the majority collected as product and a small portion reserved for reflux in the next cycle.
5. The system is repressurized to begin the next cycle.

### Example of Fixed-Bed Adsorption:

a) A mixed gas containing propylene and propane enters a fixed-bed adsorption column at a specified adsorption temperature (−30° C.-100° C., preferably 25° C.-40° C.) and pressure (0 bar-10 bar, preferably 1 bar-5 bar) with a set flow rate, where it contacts the molecular sieve with a point-sieving structure. Due to the slower adsorption rate and lower capacity for propane compared to propylene, propane preferentially breaks through the column, allowing direct recovery of propane gas at the column outlet.
b) After propane breakthrough, the feed gas continues to flow until the propylene adsorption front occupies at least two-thirds of the bed or until propylene breakthrough occurs. The adsorption process is then halted, and desorption is initiated via heating (0° C.-40° C., preferably 30° C.-40° C.), reduced pressure (0.01 bar-1 bar, preferably 0.01 bar-0.1 bar), or a combination thereof. High-purity propylene or product gas is used to purge and further concentrate the adsorbed propylene, yielding propylene gas.

The contact between the adsorbent and the propylene/propane mixture may involve temperature swing adsorption (TSA), pressure swing adsorption (PSA), or both.

The mixture may contain 99.8 vol % to 0.2 vol % propylene, 0.2 vol % to 99.8 vol % propane, and 0 vol %-10 vol % other substances. For example, a propylene: propane: others with volume ratio of 50:40:10 may be used. These additional substances have minimal impact on the separation performance of the molecular sieve.

The other substances may include at least one of methane, oxygen, nitrogen, hydrogen, ethane, and ethylene.

After the adsorbent has completed the adsorption of propylene, the adsorbent can be desorbed by at least one of reducing pressure, increasing temperature, and purging with an inert gas to obtain propylene and regenerate the adsorbent. The purity of the propylene obtained by desorption can be greater than 99.95%.

Advantages of the present invention Compared to Prior Technology:
1. The molecular sieve ZU-609 with a point-sieving structure employed in the present invention exhibits linear propylene adsorption isotherms, high pressure swing adsorption (PSA) capacity (propylene working capacity of up to 2 mmol g⁻¹ between 0.1 bar and 1 bar, see FIG. 4), and high desorption efficiency. It achieves complete regeneration under ambient conditions via inert gas purging or vacuum.
2. The present invention demonstrates exceptional propylene/propane selectivity, enabling molecular sieving of propylene and propane at ambient conditions. The propylene/propane adsorption ratio reaches 22 at 1 bar (see FIG. 5).
3. Compared to other propylene/propane sieving materials, ZU-609 exhibits a higher propylene diffusion coefficient (see FIG. 6), allowing efficient adsorption and desorption even under high gas flow rates.
4. The molecular sieve with a point-sieving structure is synthesized from widely available and low-cost raw materials under mild conditions. The method is simple, reproducible, and yields a material with excellent water/thermal stability, long lifespan, and minimal performance degradation in the presence of moisture or sulfides.
5. The separation method provided by the present invention delivers propylene gas with a purity exceeding 99.95%.

### BRIEF DESCRIPTION OF THE DRAWINGS

FIG. 1 illustrates a schematic diagram of the point-sieving pore channels in the molecular sieve ZU-609 of the present invention compared to conventional sieving pore channels.

FIG. 2 shows a structural schematic of the molecular sieve ZU-609 of the present invention.

FIG. 3 depicts the diffusion energy barrier pathway formed by propylene molecules diffusing through the pore channels of ZU-609 of the present invention.

FIG. 4 presents a comparison of pressure swing adsorption (PSA) capacities between ZU-609 of the present invention and prior art materials.

FIG. 5 displays the adsorption isotherms of propylene and propane on ZU-609 of the present invention at 298 K.

FIG. 6 compares the propylene diffusion coefficients of ZU-609 of the present invention with those of existing materials.

FIG. 7 shows the breakthrough curve from Example 2.

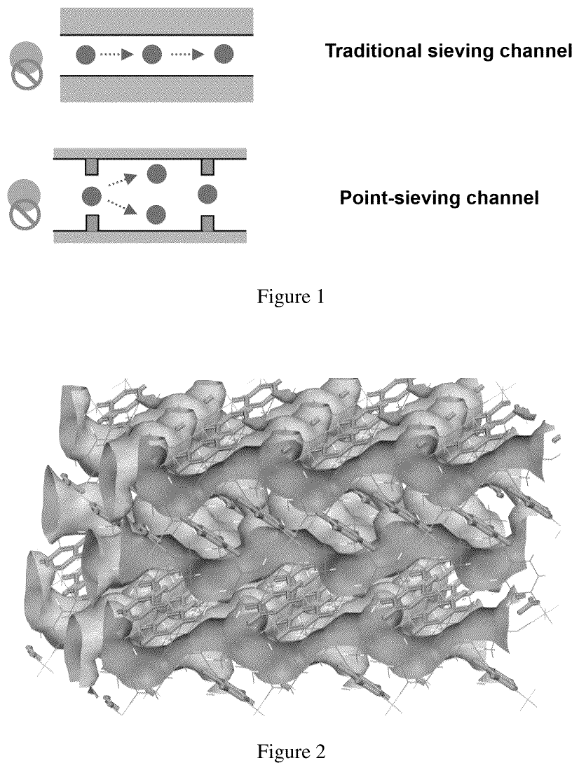

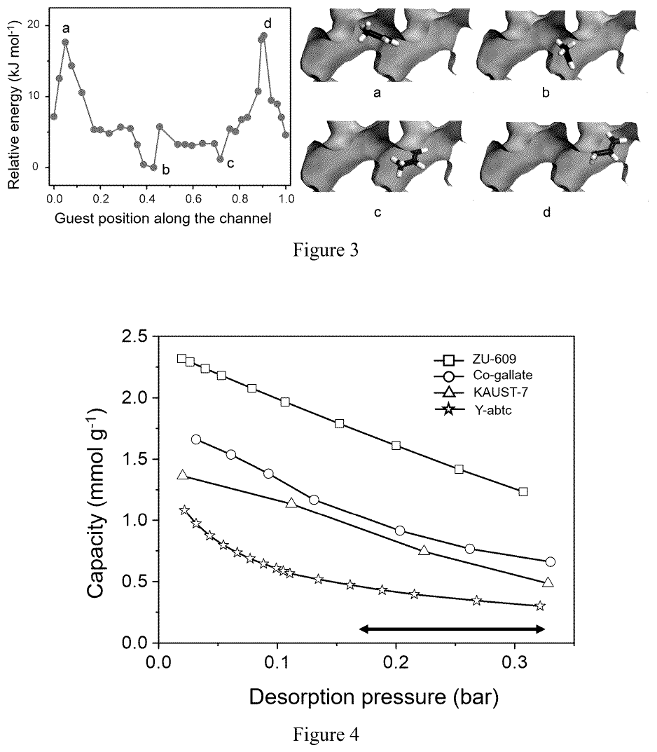

### DETAILED DESCRIPTION OF EMBODIMENTS

The present invention is further described below with reference to the accompanying drawings and specific examples. It should be understood that these examples are intended to illustrate the present invention but not to limit its scope.

### Example 1

1 mol of copper nitrate, 1 mol of sodium 1,2-ethanedisulfonate, and 1 mol of 4,4′-dipyridyl sulfide were added to 40 L of methanol and stirred at room temperature for 1 hour. After the reaction, the solid product was collected via vacuum filtration at 25° C. The sample was then activated under vacuum at 100° C. to remove guest solvent molecules from the pores, yielding the molecular sieve porous material ZU-609 with a point-sieving structure.

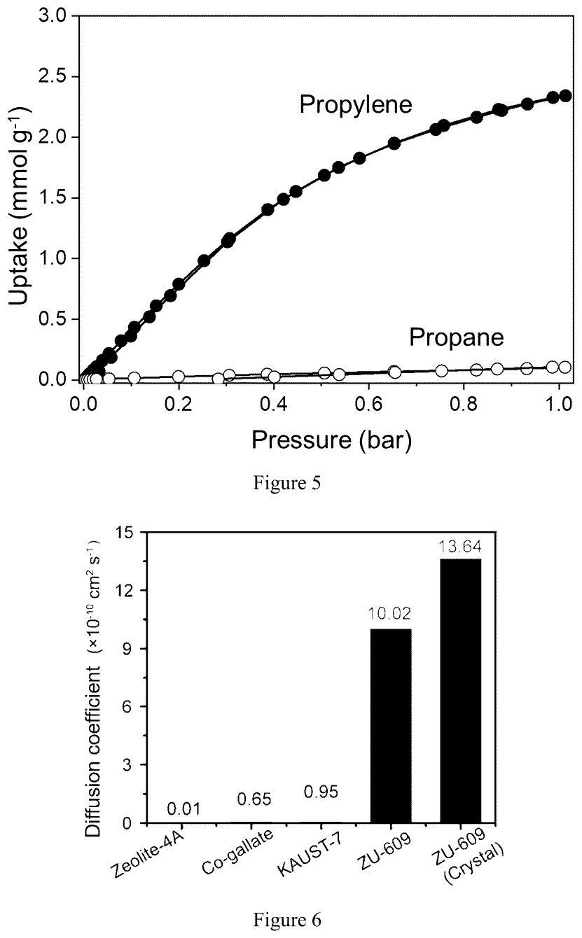

The adsorption isotherms of propylene and propane on ZU-609 at 298 K are shown in FIG. 5.

### Example 2

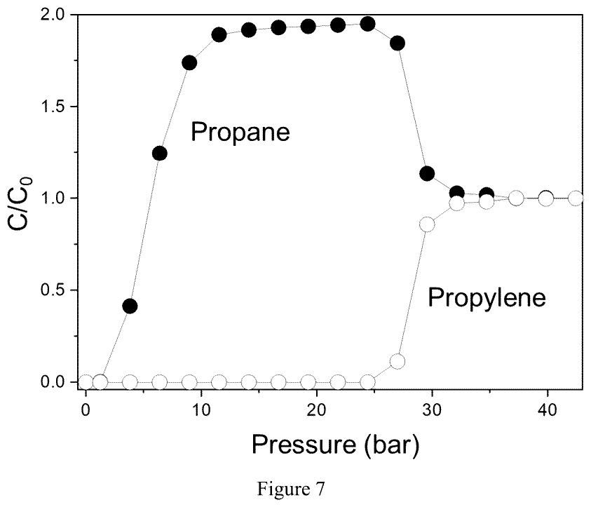

The porous material ZU-609 with a point-sieving structure was packed into a 10 cm-long fixed-bed adsorption column. A propylene/propane mixture (50:50 by volume) was introduced into the column at 25° C. and 1 bar with a flow rate of 3.0 mL/min. High-purity propane (>99.999%) was obtained at the outlet. When propylene breakthrough occurred, adsorption was halted. The column was regenerated by purging with 3.0 mL/min of nitrogen at ambient temperature, recovering propylene gas with >99.5% purity. The breakthrough curve is shown in FIG. 7.

### Example 3

The porous material ZU-609 with a point-sieving structure was packed into a 5 cm fixed-bed column. A propylene/propane mixture (40:60 by volume) was fed at 0.5 mL/min under 40° C. and 5 bar. After a certain period of adsorption, the introduction of the mixed gas was stopped after the propylene component penetrated. The propylene gas component enriched in the fixed bed was desorbed by decompression at 25° C. to 1 bar to obtain propylene gas with a purity of 99.5%.

### Example 4

The porous material ZU-609 granules with a point-sieving structure were packed into a fixed-bed column. A mixed gas (propylene:propane:ethylene:ethane=45:45:5:5 by volume) was introduced at 20 mL/min under 100° C. and 10 bar. After propylene breakthrough, desorption was conducted by heating to 30° C. and reducing the pressure to 0.1 bar. The desorbed gas was recycled for further adsorption, and propylene gas with >98% purity was obtained.

### Example 5

The porous material ZU-609 with a point-sieving structure was packed into a 50 cm fixed-bed column. A propylene/propane/methane mixture (80:15:5 by volume) was introduced at a flow rate of 100 mL/min under 5° C. and 4 bar for fixed bed adsorption. Propylene was adsorbed preferentially. When the propylene adsorption front reached two-thirds of the bed, the feed of the mixed gas was stopped. The column was depressurized until propylene breakthrough, followed by countercurrent displacement with high-purity propylene. Desorption at 40° C. and 0.5 bar yielded propylene gas with 99.99% purity and regenerated the adsorbent.

### Example 6

The porous material ZU-609 with a point-sieving structure was packed into two 100 mL fixed-bed columns. A propylene/propane mixture (99.8:0.2 by volume) was fed into Column 1 at 100 mL/min under 15° C. and 6 bar. When the adsorption front reached two-thirds of the bed, the feed was stopped. Column 1 was evacuated to 0.05 bar to recover preliminary product gas and regenerate the column. This gas was fed into Column 2, and the process was repeated. Final product gas with 99.999% purity was obtained after two adsorption-desorption cycles.

Furthermore, it should be understood that after reading the foregoing description of the present invention, those skilled in the art may make various modifications or alterations to the present invention. These equivalent forms shall likewise fall within the scope defined by the appended claims of this application.

1. A molecular sieve with a point-sieving structure, characterized by being formed through coordination bonds among 1,2-ethanedisulfonate anions, 4,4′-dipyridyl sulfide, and Cu²⁺, with a chemical formula [MSL₂]_{∞}, where M represents Cu²⁺, S represents the 1,2-ethanedisulfonate anion, L represents 4,4′-dipyridyl sulfide, and co indicates that the molecular sieve is spatially extended from multiple structural units of MSL₂;
the molecular sieve with a point-sieving structure exhibits locally contracted channels.

2. The molecular sieve with a point-sieving structure according to claim 1, characterized in that the sieving pore window has a cross-sectional dimension of 4.2×5.1 Å², and the molecular sieve possesses a large pore cavity of 7.5×8.1×11.1 Å³.

3. A method for preparing the molecular sieve with a point-sieving structure according to claim 1, characterized by mixing and reacting a Cu²⁺ source, 1,2-ethanedisulfonate salt, and 4,4′-dipyridyl sulfide in a reaction solvent to obtain the molecular sieve with a point-sieving structure.

4. The method according to claim 3, characterized in that: the reaction solvent is water and/or an organic solvent, wherein the organic solvent comprises methanol;
the Cu²⁺ source comprises at least one of chloride or nitrate compounds;
the 1,2-ethanedisulfonate salt comprises sodium 1,2-ethanedisulfonate;
the molar ratio of Cu²⁺: 1,2-ethanedisulfonate anion: 4,4′-dipyridyl sulfide is 1:0.5-3:1-5;
the ratio of Cu²⁺ source to reaction solvent is 1 mol:35-45 L;
solid-liquid separation is performed after the reaction, and the solid is treated to remove solvent molecules from the pores, yielding the molecular sieve with a point-sieving structure.

5. A method for adsorption of propylene comprising the step of utilizing the molecular sieve with a point-sieving structure according to claim 1.

6. A method for adsorptive separation of propylene and propane, characterized by contacting a mixture containing propylene and propane with the molecular sieve according to claim 1 as an adsorbent, wherein the adsorbent selectively adsorbs propylene while excluding propane, thereby achieving separation of propylene and propane.

7. The method according to claim 6, characterized in that the contact method with propylene and propane is selected from one or more of fixed-bed adsorption, moving-bed adsorption, multi-tower pressure swing adsorption, or a combination thereof.

8. The method according to claim 6, characterized in that an adsorption process involves temperature swing adsorption, pressure swing adsorption, or both.

9. The method according to claim 6, characterized in that the mixture contains 99.8 vol %-0.2 vol % propylene, 0.2 vol %-99.8 vol % propane, and 0 vol %-10 vol % other substances, other substances comprise at least one of methane, oxygen, nitrogen, hydrogen, ethane, or ethylene.

10. The method according to claim 6, characterized in that after propylene adsorption, the adsorbent is regenerated via desorption under reduced pressure, heating, inert gas purging, or a combination thereof, to recover propylene.
